(19) United States
(12) Patent Application Publication
(10) Pub. No.: US 2026/0184667 A1
(43) Pub. Date: Jul. 2, 2026

(54) CGMP-COMPLIANT AUTOMATED [18F]FSPG PRODUCTION FOR CLINICAL EXAMINATION

(71) Applicant: BOARD OF REGENTS, THE UNIVERSITY OF TEXAS SYSTEM, Austin, TX (US)

(72) Inventors: Mai LIN, Houston, TX (US); Henry Charles MANNING, Houston, TX (US)

(21) Appl. No.: 18/867,737

(22) PCT Filed: May 22, 2023
(86) PCT No.: PCT/US2023/023108

§ 371 Date: Nov. 20, 2024

Related U.S. Application Data
(60) Provisional application No. 63/344,902, filed on May 23, 2022.

(51) Int. Cl. C07C 227/18; A61K 51/04; C07B 59/00; C07C 227/40

(52) U.S. Cl. CPC C07C 227/18; A61K 51/0406; C07B 59/001; C07C 227/40; A61K 2123/00; C07B 2200/05; C07B 2200/07

(57) ABSTRACT

According to various aspects of this disclosure, the present disclosure relates to an automated process for the radiosynthesis of [¹⁸F]FSPG, a positron emission tracer. The automated optimized process of the present invention enables the centralized and large-scale production of [¹⁸F]FSPG for commercial use.

### CROSS-REFERENCE TO RELATED APPLICATIONS

This application claims the benefit of U.S. Provisional Application No. 63,344,902, filed May 23, 2022, which is incorporated by reference herein in its entirety.

### FIELD OF THE DISCLOSURE

The present disclosure relates to the field of methods and devices for automated synthesis of compounds suitable for use as in vivo imaging agents and methods of using the same.

### BACKGROUND

Radiolabeled compounds for use as in vivo imaging agents are currently typically prepared by means of an automated synthesis apparatus (alternatively, a “radiosynthesizer”). Such automated synthesis apparatuses are commercially available from a range of suppliers, including: GE Healthcare; CTI Inc.; Ion Beam Applications S.A. (Chemin du Cyclotron 3, B-1348 Louvain-La-Neuve, Belgium); Raytest (Germany) and Bioscan (USA). The radiochemistry takes place in a “cassette” or “cartridge” designed to be removed and fitted interchangeably onto the apparatus, in such a way that mechanical movement of moving parts of the apparatus controls the operation of the cassette. Suitable cassettes may be provided as part of a kit that is assembled onto the apparatus in a number of steps, or may be provided as a single piece that is attached in a single step, thereby reducing the risk of human error. The single piece arrangement is generally a disposable single-use cassette, which comprises all the reagents, eluents, reaction vessels, and apparatus necessary to carry out the preparation of a given batch of radiopharmaceutical.

(S)-4-(3-¹⁸F-fluoropropyl)-L-glutamic acid ([¹⁸F]FSPG) is a positron emission tomography (PET) tracer that specifically targets the cystine/glutamate antiporter (Xc⁻)—a biomarker that is frequently overexpressed in cancer and several neurological disorders. Pilot studies examining dosimetry and biodistribution in healthy volunteers and tumor detection in patients with non-small cell lung cancer, hepatocellular carcinoma, and brain tumors showed promising results. In particular, low background uptake in the brain, lung, and bowel was observed that further leads to excellent imaging contrasts of [¹⁸F]FSPG PET.

However, the clinical adoption of this radiotracer lags due to a lack of a reliable and cGMP-compliant automated processes for [¹⁸F]FSPG production. Thus, a need remains for a robust, cGMP-compliant process to enable the large-scale production necessary for clinical and commercial use of the [¹⁸F]FSPG radiotracer.

### BRIEF SUMMARY

In some aspects of the disclosure are directed to an automated process for the synthesis of (S)-4-(3-¹⁸F-fluoropropyl)-L-glutamic acid (¹⁸F-FSPG) in a cassette, wherein the process comprises the following steps:
a. Dissolving a (S)-4-(3-¹⁸F-fluoropropyl)-L-glutamic acid precursor in anhydrous acetonitrile and adding the resulting solution to a reaction vessel comprising radiolabeled fluoride (¹⁸F);
b. Heating the reaction vessel at 100° C. to 110° C. (e.g., for 5 minutes to 15 minutes) to provide a crude intermediate product;
c. Adding a first aliquot of an acid to the reaction vessel and heating at 100° C. to 110° C. (e.g., for 2 to 10 minutes) followed by adding an aliquot of a base to the reaction vessel and heating at 60° C. to 80° C. (e.g., for 5 to 15 minutes) to remove protecting groups to provide a crude ¹⁸F-FSPG;
d. Acidifying the crude ¹⁸F-FSPG;
e. Loading the acidified crude ¹⁸F-FSPG onto one or more reverse-phase solid phase extraction cartridges;
f. Eluting the ¹⁸F-FSPG off the one or more reverse-phase solid phase extraction cartridges;
g. Purifying the ¹⁸F-FSPG through one or more normal-phase solid phase extraction cartridges; and
wherein each of (a)-(g) are within the cassette.

In some aspects, the ¹⁸F-FSPG is eluted off the one or more reverse-phase solid phase extraction cartridges with a buffer. In some aspects, the buffer is phosphate-buffered saline.

In some aspects, the acid in step (c) is selected from the group consisting of sulfuric acid, hydrochloric acid, and trifluoroacetic acid. In some aspects, the acid in step (c) is sulfuric acid or trifluoroacetic acid. In some aspects, the acid in step (c) is sulfuric acid. In some aspects, the acid in step (c) is hydrochloric acid. In some aspects, the acid in step (c) is trifluoroacetic acid.

In some aspects, the base in step (c) is sodium hydroxide or potassium hydroxide. In some aspects, the base in step (c) is sodium hydroxide. In some aspects, the base in step (c) is potassium hydroxide.

In some aspects, the one or more reverse-phase solid phase extraction cartridges are washed with water to remove side products before step (f).

In some aspects, the crude ¹⁸F-FSPG is acidified with a second aliquot of an acid in step (d). In some aspects, the crude ¹⁸F-FSPG is acidified with a second aliquot of sulfuric acid in step (d).

In some aspects, the ¹⁸F-FSPG is purified through one or more normal-phase solid phase extraction cartridges outside of the cassette. In some aspects, the ¹⁸F-FSPG product is further sterilized through a sterilizing filter to produce a final ¹⁸F-FSPG composition.

In some aspects, the final purified ¹⁸F-FSPG has a greater than 90%, 95%, 96%, 97%, or 98% radiochemical purity.

In some aspects, the process produces ¹⁸F-FSPG with a non-decay corrected radiochemical yield of at least 10%. In some aspects, the process produces ¹⁸F-FSPG with a non-decay corrected radiochemical yield of 10% to 40%. In some aspects, the process produces ¹⁸F-FSPG with a non-decay corrected radiochemical yield of 13% to 35%. In some aspects, the process produces ¹⁸F-FSPG with a non-decay corrected radiochemical yield of 18% to 32%.

In some aspects, the reaction product is loaded onto the reverse-phase solid phase extraction with a flow rate of 8 ml/min to 20 ml/min and eluted off the reverse-phase solid phase extraction cartridge with a flow rate of 4 ml/min to 14 ml/min. In some aspects, the reaction product is loaded onto the reverse-phase solid phase extraction with a flow rate of 14 ml/min and eluted off the reverse-phase solid phase extraction cartridge with a flow rate of 10 ml/min.

In some aspects, the reverse-phase solid phase extraction cartridge comprises an MCX Oasis cartridge. In some aspects, the [¹⁸F]FSPG is eluted off the reverse-phase solid phase extraction cartridge in the same direction as the crude [¹⁸F]FSPG was loaded. In some aspects, the normal-phase solid extraction cartridges comprise an alumina Sep-Pak cartridge and/or an ENVI-carb or Hypercarb cartridge. In some aspects, the normal-phase solid extraction cartridges comprises an alumina Sep-Pak cartridge. In some aspects, the normal-phase solid extraction cartridges comprises an ENVI-carb cartridge. In some aspects, the normal-phase solid extraction cartridges comprises a Hypercarb cartridge.

In some aspects, the FSPG precursor has one of the following structure(s):

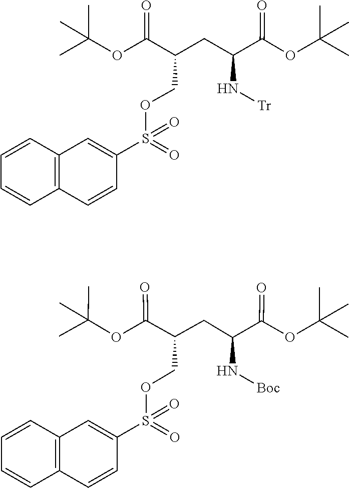

In some aspects, the ¹⁸F-FSPG has a starting activity of about 10 to 110 Gbq.

In some aspects, the final ¹⁸F-FSPG composition has a concentration of about 185 to 1850 MBq/ml.

In some aspects, the final ¹⁸F-FSPG composition is produced in less than 40 minutes from the start of synthesis (e.g., 5 to 40 minutes, 10 to 40 minutes, 15 to 40 minutes, 5 to 35 minutes, 10 to 30 minutes, 15 to 30 minutes from the start of synthesis).

In some aspects, the final ¹⁸F-FSPG composition has a pH of 6.5 to 7.5.

In some aspects, the radiolabeled fluoride is prepared by a method comprising:
a. Irradiating ¹⁸O water to produce a solution of ¹⁸F in ¹⁸O water;
b. Loading the ¹⁸F solution onto a quaternary ammonium anion exchange cartridge pre-conditioned with potassium bicarbonate;
c. Eluting ¹⁸F from the cartridge into the reaction vessel with an eluent of potassium carbonate/4,7,13,16,21,24-hexaoxa-1,10-diazabicyclo[8.8.8]hexacosane in 50% water/acetonitrile solution; and
d. Heating the reaction vessel to remove water.

In some aspects, a composition comprising ¹⁸F-FSPG is prepared according to any process of the disclosure.

In some aspects, the ¹⁸F-FSPG composition does not undergo radiolysis for at least 1 hour, 2 hours, 3 hours, 4 hours, 5 hours, 6 hours, 7 hours, or 8 hours after synthesis when stored at room temperature conditions.

In some aspects, the ¹⁸F-FSPG composition maintains a greater than 90%, 95%, 97%, 98%, or 99% radiochemical purity for at least 5 hours, at least 6 hours, at least 7 hours, or at least 8 hours after storage at room temperature conditions. In some aspects, the ¹⁸F-FSPG composition maintains a greater than 90%, 95%, 97%, 98%, or 99% radiochemical purity for at least 1 hours, 2 hours, 3 hours, 4 hours, 5 hours, 6 hours, 7 hours, or 8 hours after storage at room temperature.

### DESCRIPTION OF FIGURES

FIG. 1 illustrates an exemplary cassette suitable for synthesizing [¹⁸F]FSPG.

FIG. 2 illustrates an exemplary workflow for synthesizing [¹⁸F]FSPG using a single-use cassette (such as the illustrated in FIG. 1).

FIG. 3 illustrates the stability of a final [¹⁸F]FSPG composition when stored at room temperature.

### DETAILED DESCRIPTION

The present invention concerns devices and methods for the automated synthesis of an [¹⁸F]-labeled compounds, e.g., [¹⁸F]FSPG, which are suitable for use as in vivo imaging agent for positron emission tomography (PET), using a disposable cassette.

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those disclosed herein can be used in practice or testing of the present disclosure, suitable methods and materials are disclosed below. The materials, methods and examples are illustrative only and are not intended to be limiting. Other features and advantages of the disclosure will be apparent from the detailed description and from the claims.

### I. Definitions

In order to further understand this disclosure, the following terms and definitions are provided.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

Throughout this disclosure, various aspects of this disclosure are presented in a range format. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values, which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are disclosed herein with the language “comprising,” otherwise analogous aspects disclosed in terms of “consisting of” and/or “consisting essentially of” are also provided.

As used herein, the term “cassette” is means a component of an apparatus (e.g., an automated synthesis apparatus) designed to fit removably onto the apparatus. In some aspects, the cassette can be removed in such a way that mechanical movement of moving parts of the apparatus (e.g., automated synthesizer apparatus) controls the operation of the cassette from outside the cassette, i.e., externally. In some aspects, the cassette can be for single use.

The term “single-use” as used in the context of a cassette of the present disclosure means that the cassette is intended to be used for one use or duration (e.g., for a single or plurality of batches) prior to disposal.

As used herein, the term “selectively fluidly connected” means that it is possible to select whether or not a fluid can pass to and/or from a feature to another feature of the cassette, e.g. by use of a suitable valve. In some aspects, a suitable valve comprises a 3-way valve having three ports and means to put any two of the three associated ports in fluid communication with each other while fluidly isolating the third port. In some aspects, a suitable valve comprises a stopcock valve comprising a rotatable stopcock. In some aspects, the components of the cassette are selectively fluidly connected along a common pathway.

The term “common pathway” as used herein refers to a fluid pathway to which the certain components of a system (e.g., an automated synthesis apparatus) and/or of a cassette of the present disclosure are selectively fluidly connected. In some aspects, the common pathway is a linear fluid pathway. In some aspects, the common pathway is made from a rigid pharmaceutical grade polymeric material that is resistant to radiation. Non-limiting examples of suitable such materials include polypropylene, polyethylene, polysulfone, and Ultem®. In some aspects, said common pathway is made from polypropylene or polyethylene.

As used herein the term “automated synthesis apparatus” means an automated module based on the principle of unit operations, e.g., as described by Satyamurthy et al (1999 Clin Positr Imag; 2 (5): 233-253), which is incorporated herein by reference. The term “unit operations” means that complex processes are reduced to a series of more basic or simple operations or reactions, which can be applied to a range of materials. Such automated synthesis apparatuses are preferred for the methods of the present disclosure especially when a radiopharmaceutical composition is desired to be prepared. Such automated synthesis apparatuses are commercially available from a range of suppliers (Satyamurthy et al, above), including: GE Healthcare; CTI Inc; Ion Beam Applications S.A. (Chemin du Cyclotron 3, B-1348 Louvain-LaNeuve, Belgium); Raytest (Germany) and Bioscan (USA). In certain aspects, the automated synthesis apparatuses are designed to be employed in a suitably configured radioactive work cell, or “hot cell”, which provides suitable radiation shielding to protect the operator from potential radiation dose, as well as ventilation to remove chemical and/or radioactive vapors. Using a cassette, the automated synthesis apparatus has the flexibility to make a variety of different radiopharmaceuticals with minimal risk of cross-contamination, by allowing for the changing of the cassette. This approach also has the advantages of simplified set-up, hence reduced risk of operator error, improved GMP (good manufacturing practice) compliance, multi-tracer capability, rapid change between production runs, pre-run automated diagnostic checking of the cassette and reagents, automated barcode cross-check of chemical reagents vs the synthesis to be carried out, reagent traceability, single-use and hence no risk of cross-contamination, tamper and abuse resistance.

A “reaction vessel” in the context of the present disclosure is a container or receptacle of a cassette where the reactants and reagents required for the PET tracer synthesis can be sent and the product(s) removed in an appropriate order. In some aspects, the reaction vessel has an internal volume suitable for containing the reactants and reagents and is made from pharmaceutical grade materials resistant to radiation. In some aspects, the reaction vessel can be heated to catalyze reactions inside, increase reaction speeds, or evaporate solvents. In some aspects, the reaction vessel is heated by a reactor heater (e.g., as a component of the automated synthesis apparatus, e.g., a GE FASTlab apparatus).

An “aliquot” in the context of a method of the present disclosure is a sufficient quantity of a particular reagent for use during a step of the method (e.g., in a synthesis step of a PET tracer).

As used herein, a “precursor compound” is a non-radioactive derivative of a radiolabeled compound. In some aspects, the precursor compound is designed so that chemical reaction with a convenient chemical form of a detectable label occurs site-specifically in the minimum number of steps to give the desired radiolabeled compound. In certain aspects, for site-specific labelling, a precursor compound can have protecting groups. In some aspects, the precursor compound is synthetic and can conveniently be obtained having good chemical purity.

The term “protecting group” refers to a group which inhibits or suppresses undesirable chemical reactions of a molecule, but which is designed to be sufficiently reactive that it may be removed (e.g., cleaved) from the functional group of the molecule to obtain a desired product under conditions that do not modify the rest of the molecule. Protecting groups and methods for their removal (i.e. “deprotection”) are well known to those skilled in the art and are described in ‘Protective Groups in Organic Synthesis’, Theorodora W. Greene and Peter G. M. Wuts, (Fourth Edition, John Wiley & Sons, 2007), which is incorporated herein by reference. In some aspects, a deprotecting agent is selected from HCl, NaOH, H₃PO₄, trifluoroacetic acid, and H₂SO₄. In some aspects, more than one deprotecting agent is required to remove more than one protecting group on the precursor compound. In some aspects, the deprotecting agent is NaOH. In some aspects, the deprotecting agent is H₂SO₄. In some aspects, the deprotecting agent is trifluoroacetic acid.

The term “reagent” as used herein refers to solvents and/or reactants used in the synthesis of a molecule, compound, or product (e.g., a [¹⁸F]-labeled PET tracer). In some aspects, the reagent or reagents are stored in a reagent vial.

The term “reagent vial” refers to a receptacle or vial containing at least one reagent for use in a method, apparatus, or cassette disclosed herein, e.g., in the production of a [¹⁸F]-labeled PET tracer. In some aspects, the reagent vial is sufficient for containing enough reagent(s) for the production of a desired batch or plurality of batches.

The term “sufficient” means a suitable amount, e.g., of a reagent to allow for completion of one or more steps or processes that require the reagent.

The term “solid phase extraction” or “SPE” refers to a sample preparation process by which compounds in a solution are separated from each other based on their respective affinities for a solid (the “solid phase”, or “stationary phase”) through which the sample is passed and the solvent (the “mobile phase” or “liquid phase”) in which they are dissolved. Using the SPE process, a compound of interest is either retained on the solid phase or in the mobile phase. The portion that passes through the solid phase can be collected or discarded, depending on whether it contains the compound of interest. If the portion retained on the stationary phase includes the compound of interest, it can then be removed from the stationary phase for collection in an additional step, in which the stationary phase is rinsed with another solution known as an “eluent.” For the present disclosure, SPE can be suitably carried out using an “SPE cartridge” (also referred to as an “SPE column”), which is readily available commercially and is typically in the form of a syringe-shaped column packed with solid phase. In some aspects, the solid phases are based on silica that has been bonded to a specific functional group, e.g. hydrocarbon chains of variable length (suitable for reverse-phase SPE), quaternary ammonium or amino groups (suitable for anion exchange), and sulfonic acid or carboxyl groups (suitable for cation exchange).

As used herein, the term “eluting” refers to passing a solution through a solid phase, e.g., via an SPE cartridge, with the aim to release a compound or compounds of interest that has or have been bound to the solid phase.

As used herein, the term “trapping” refers to the process wherein a particular compound or compounds binds to the solid phase, e.g., of an SPE cartridge.

The term “purifying” or “purification” as used herein may be taken to mean a process to obtain a substantially pure molecule, compound or product (e.g., a [¹⁸F]-labeled compound). The term “substantially” refers to the complete or nearly complete extent or degree of an action, characteristic, property, state, structure, item, or result (e.g., suitable for use as a PET tracer). The term “substantially pure” can be taken to mean completely pure or nearly completely pure or free of contaminants that would interfere with the intended purpose (e.g., unused reagents or by-products).

The term “suitable for use as a PET tracer” means that the [¹⁸F]-labeled compound is suitable for intravenous administration to a mammalian subject followed by PET imaging to obtain one or more useful images of the location and/or distribution of the [¹⁸F]-labeled compound.

The term “sterilized” refers to the process of removing potential microbial contaminants from a molecule, compound, product, or a composition comprising the same (e.g., a [¹⁸F]FSPG solution).

### II. PET Tracers

Certain aspects of the disclosure are directed to components (e.g., cassettes), apparatuses (e.g., automated synthesis apparatus) and methods of using the same for the synthesis of a positron emission tomography (PET) tracer (or radiotracer). In some aspects, the PET tracer is a (S)-4-(3-¹⁸F-fluoropropyl)-L-glutamic acid ([¹⁸F]FSPG) radiotracer ([¹⁸F]FSPG radiotracer). In some aspects, the [¹⁸F]-labeled compound obtained by the methods of the disclosure is substantially pure. In some aspects, the [¹⁸F]-labeled compound obtained by the methods of the disclosure is completely pure. In some aspects, the [¹⁸F]-labeled compound obtained by the methods of the disclosure is nearly completely pure.

In some aspects, the disclosure provides for production [¹⁸F]FSPG with a radiochemical purity of at least 90%. In some aspects, the disclosure provides for production of [¹⁸F]FSPG with a radiochemical purity of at least 91%. In some aspects, the disclosure provides for production of [¹⁸F]FSPG with a radiochemical purity of at least 92%. In some aspects, the disclosure provides for production of [¹⁸F]FSPG with a radiochemical purity of at least 93%. In some aspects, the disclosure provides for production of [¹⁸F]FSPG with a radiochemical purity of at least 94%. In some aspects, the disclosure provides for production of [¹⁸F]FSPG with a radiochemical purity of at least 95%. In some aspects, the disclosure provides for production of [¹⁸F]FSPG with a radiochemical purity of at least 96%. In some aspects, the disclosure provides for production of [¹⁸F]FSPG with a radiochemical purity of at least 97%. In some aspects, the disclosure provides for production of [¹⁸F]FSPG with a radiochemical purity of at least 98%. In some aspects, the disclosure provides for production of [¹⁸F]FSPG with a radiochemical purity of 90% to 99%. In some aspects, the disclosure provides for production of [¹⁸F]FSPG with a radiochemical purity of 91% to 98%. In some aspects, the disclosure provides for production of [¹⁸F]FSPG with a radiochemical purity of 92% to 97%. In some aspects, the disclosure provides for production of [¹⁸F]FSPG with a radiochemical purity of 93% to 97%. In some aspects, the disclosure provides for production of [¹⁸F]FSPG with a radiochemical purity of 93% to 96%.

In some aspects, the disclosure provides for production [¹⁸F]FSPG with a radiochemical yield of 10% to 40% (non-decay corrected). In some aspects, the disclosure provides for production [¹⁸F]FSPG with a radiochemical yield of 10% to 35% (non-decay corrected). In some aspects, the disclosure provides for production [¹⁸F]FSPG with a radiochemical yield of 13% to 35% (non-decay corrected). In some aspects, the disclosure provides for production [¹⁸F]FSPG with a radiochemical yield of 18% to 32% (non-decay corrected). In some aspects, the disclosure provides for production [¹⁸F]FSPG with a radiochemical yield of 20% to 35% (non-decay corrected). In some aspects, the disclosure provides for production [¹⁸F]FSPG with a radiochemical yield of 13% to 35% (non-decay corrected). In some aspects, the disclosure provides for production [¹⁸F]FSPG with a radiochemical yield of 25% to 35% (non-decay corrected). In some aspects, the disclosure provides for production [¹⁸F]FSPG with a radiochemical yield of at least 10% (non-decay corrected). In some aspects, the disclosure provides for production [¹⁸F]FSPG with a radiochemical yield of at least 15% (non-decay corrected). In some aspects, the disclosure provides for production [¹⁸F]FSPG with a radiochemical yield of at least 20% (non-decay corrected). In some aspects, the disclosure provides for production [¹⁸F]FSPG with a radiochemical yield of at least 25% (non-decay corrected). In some aspects, the disclosure provides for production [¹⁸F]FSPG with a radiochemical yield of at least 30% (non-decay corrected). In some aspects, the disclosure provides for production [¹⁸F]FSPG with a radiochemical yield of at least 35% (non-decay corrected).

In some aspects, the disclosure provides for production of [¹⁸F]FSPG that does not undergo radiolysis after 1 hour when stored at room temperature. In some aspects, the disclosure provides for production of [¹⁸F]FSPG that does not undergo radiolysis after 2 hours when stored at room temperature. In some aspects, the disclosure provides for production of [¹⁸F]FSPG that does not undergo radiolysis after 3 hours when stored at room temperature. In some aspects, the disclosure provides for production of [¹⁸F]FSPG that does not undergo radiolysis after 4 hours when stored at room temperature. In some aspects, the disclosure provides for production of [¹⁸F]FSPG that does not undergo radiolysis after 5 hours when stored at room temperature.

In some aspects, the disclosure provides for production of [¹⁸F]FSPG with a radiochemical purity of at least 90% after 1 hour, 2 hours, 3 hours, 4 hours, 5 hours, 6 hours, 7 hours, or 8 hours of storage at room temperature. In some aspects, the disclosure provides for production of [¹⁸F]FSPG with a radiochemical purity of at least 91% after 1 hour, 2 hours, 3 hours, 4 hours, 5 hours, 6 hours, 7 hours, or 8 hours of storage at room temperature. In some aspects, the disclosure provides for production of [¹⁸F]FSPG with a radiochemical purity of at least 92% after 1 hour, 2 hours, 3 hours, 4 hours, 5 hours, 6 hours, 7 hours, or 8 hours of storage at room temperature. In some aspects, the disclosure provides for production of [¹⁸F]FSPG with a radiochemical purity of at least 93% after 1 hour, 2 hours, 3 hours, 4 hours, 5 hours, 6 hours, 7 hours, or 8 hours of storage at room temperature. In some aspects, the disclosure provides for production of [¹⁸F]FSPG with a radiochemical purity of at least 94% after 1 hour, 2 hours, 3 hours, 4 hours, 5 hours, 6 hours, 7 hours, or 8 hours of storage at room temperature. In some aspects, the disclosure provides for production of [¹⁸F]FSPG with a radiochemical purity of at least 95% after 1 hour, 2 hours, 3 hours, 4 hours, 5 hours, 6 hours, 7 hours, or 8 hours of storage at room temperature. In some aspects, the disclosure provides for production of [¹⁸F]FSPG with a radiochemical purity of at least 96% after 1 hour, 2 hours, 3 hours, 4 hours, 5 hours, 6 hours, 7 hours, or 8 hours of storage at room temperature. In some aspects, the disclosure provides for production of [¹⁸F]FSPG with a radiochemical purity of at least 97% after 1 hour, 2 hours, 3 hours, 4 hours, 5 hours, 6 hours, 7 hours, or 8 hours of storage at room temperature. In some aspects, the disclosure provides for production of [¹⁸F]FSPG with a radiochemical purity of at least 98% after 1 hour, 2 hours, 3 hours, 4 hours, 5 hours, 6 hours, 7 hours, or 8 hours of storage at room temperature. In some aspects, the disclosure provides for production of [¹⁸F]FSPG with a radiochemical purity of at least 95% after 1 hour, 2 hours, 3 hours, 4 hours, 5 hours, 6 hours, 7 hours, or 8 hours of storage at room temperature. In some aspects, the disclosure provides for production of [¹⁸F]FSPG with a radiochemical purity of at least 95% after 8 hours of storage at room temperature.

In some aspects, the final [¹⁸F]FSPG composition obtained by the methods of the disclosure is provided as [¹⁸F]FSPG in a phosphate-buffered saline solution. In some aspects, the final [¹⁸F]FSPG composition obtained by the methods of the disclosure has a pH of about 6 to 8. In some aspects, the final [¹⁸F]FSPG composition obtained by the methods of the disclosure has a pH of about 6.5 to 7.5.

In some aspects, the final [¹⁸F]FSPG composition obtained by the methods of the disclosure has concentration of 185 to 2035 MBq/ml (5 to 55 mCi/ml). In some aspects, the final [¹⁸F]FSPG composition obtained by the methods of the disclosure has concentration of 185 to 1850 MBq/ml (5 to 50 mCi/ml). In some aspects, the final [¹⁸F]FSPG [¹⁸F]FSPG obtained by the methods of the disclosure has concentration of 260 to 1925 MBq/ml (7 to 52 mCi/ml). In some aspects, the final [¹⁸F]FSPG [¹⁸F]FSPG obtained by the methods of the disclosure has concentration of 260 to 1850 MBq/ml (7 to 50 mCi/ml).

### III. Cassettes

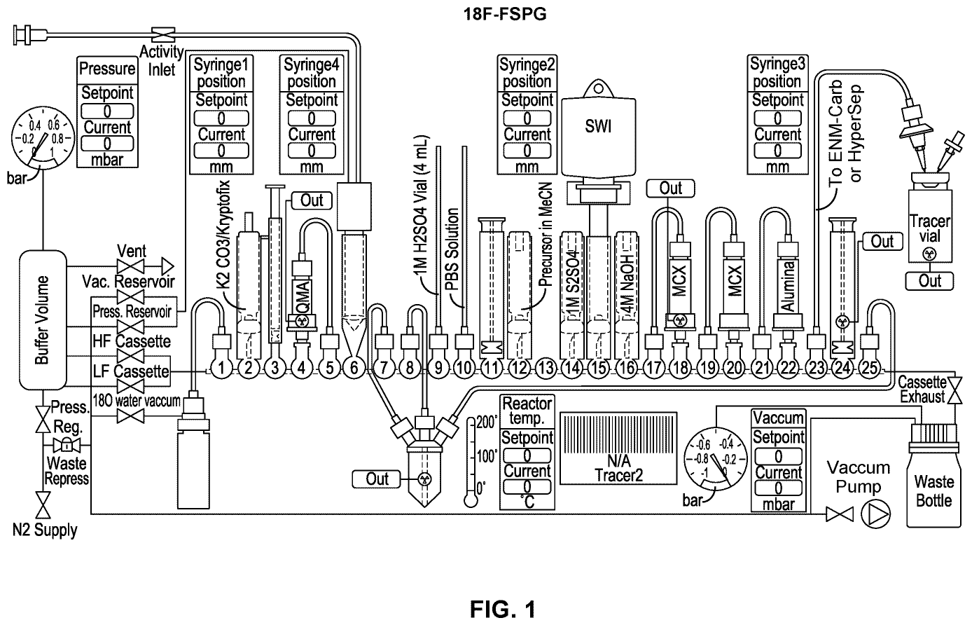

In some aspects, the cassette of the disclosure is a disposable single-piece type of cassette pre-loaded with reagents comprising a linear array of valves, each valve linked to a port where containers or vials can be attached (e.g., by either needle puncture of an inverted septum-sealed vial, or by gas-tight, marrying joints). In one aspect, each valve is a 3-way valve. In some aspects, the cassette has 25 identical 3-way valves in a linear array, an example of which is shown in FIG. 1. In one aspect, each valve is a stopcock valve comprising a rotatable stopcock. In certain aspects, each valve has a male-female joint which interfaces with a corresponding moving arm of the automated synthesis apparatus. External rotation of the arm thus controls the opening or closing of the valve when the cassette is attached to the automated synthesis apparatus. Additional moving parts of the automated synthesis apparatus can be designed to clip onto syringe plunger tips, and thus raise or depress syringe barrels. The cassette is versatile, in some aspects having several positions where reagents can be attached, and several suitable for attachment of a syringe vial of reagents or chromatography columns. In some aspects, the cassette comprises a reaction vessel, generally configured such that three (3) or more ports of the cassette are connected thereto to permit transfer of reagents or solvents from various ports on the cassette, as well as to permit transfer of reaction products from the reaction vessel to various ports on the cassette.

In some aspects, the reagent vials are made from a rigid pharmaceutical grade polymer resistant to radiation. In some aspects, a suitable reagent contained in a reagent vial can include ethanol, acetonitrile, deprotecting agents, buffers, or any combination thereof. In some aspects, the reagent comprises a deprotecting agent. In some aspects, the reagent comprises a buffer. In some aspects, the buffer is based on a weak acid, for example, selected from citrate, phosphate, acetate and ascorbate. In some aspects, the buffer is phosphate-buffered saline (PBS). In some aspects, each reagent is contained in a separate reagent vial. For example, in some aspects, a single-use cassette of the present disclosure comprises a reagent vial comprising acetonitrile, another reagent vial comprising NaOH, one or more reagent vials comprising H₂SO₄, and another reagent vial comprising a PBS.

In certain aspects, the cassettes are designed to be suitable for radiopharmaceutical manufacture and are therefore manufactured from materials which are of pharmaceutical grade as well as resistant to radiolysis. In one aspect, the single-use cassette is suitable for use with a GE Healthcare FASTlab™ automated synthesis apparatus. In some aspects, the various elements of the cassette are selectively fluidly connected.

FIG. 1 illustrates a cassette suitable for synthesizing [¹⁸F]FSPG on an automated synthesis apparatus (e.g., GE Healthcare's FASTlab™).

In some aspects of the disclosure, position 1 on the cassette contains tubing connected to a vial or container. In certain aspects of the disclosure, the tubing at position 1 is silicone tubing. In certain aspects of the disclosure, the tubing at position 1 is 12-16 cm (e.g., about 14 cm) silicone tubing.

In some aspects of the disclosure, position 2 on the cassette contains a vial or container comprising eluent. In some aspects of the disclosure, the vial or container at position 2 comprises an eluent solution of K₂CO₃/Kryptofix™ 222 in about 50% water/acetonitrile.

In some aspects of the disclosure, positions 3, 11, and 24 on the cassette each contain a syringe. In some aspects of the disclosure, moving parts of the automated synthesis apparatus designed to clip onto syringe plunger tips, and thus raise or depress the syringe barrels.

In some aspects of the disclosure, position 4 on the cassette contains a SPE cartridge. In some aspects, the SPE cartridge is an anion exchange SPE cartridge. In some aspects, the anion exchange SPE cartridge is a quaternary ammonium anion exchange (QMA) SPE cartridge. In some aspects of the disclosure, the anion exchange SPE cartridge is pre-conditioned with eluent. In some aspects, the anion exchange SPE cartridge is pre-conditioned with acetonitrile. In some aspects, the anion exchange SPE cartridge is a QMA SPE cartridge that has been pre-conditioned with acetonitrile. In some aspects, the SPE cartridge at position 4 is connected to tubing at position 5 of the cassette.

In certain aspects of the disclosure, the tubing at position 5 is silicone tubing. In certain aspects of the disclosure, the tubing at position 5 is 12-16 cm (e.g., about 14 cm) silicone tubing.

In some aspects of the disclosure, position 6 on the cassette contains a means for transferring [¹⁸F]fluoride to the cassette. In some aspects, the means for conical reservoir transferring [¹⁸F]fluoride to the cassette is a conical reservoir.

In some aspects of the disclosure, positions 7, 8, and 25 on the cassette contain tubing connected to three ports on the reaction vessel. In some aspects, the tubing at position 7 is connected to the left port on the reaction vessel, the tubing at position 8 is connected to the center port on the reaction vessel, and the tubing at position 25 is connected to the right port on the reaction vessel. In certain aspects of the disclosure, the tubing at positions 7, 8, and/or 25 is silicone tubing. In certain aspects of the disclosure, the tubing at positions 7 and/or 8 is 12-16 cm (e.g., about 14 cm) silicone tubing. In certain aspects of the disclosure, the tubing at position 25 is 40-44 cm (e.g., about 42 cm) silicone tubing.

In some aspects of the disclosure, positions 9 and 10 on the cassette contains tubing connected to vials or containers. In certain aspects of the disclosure, the tubing at positions 9 and/or 10 is silicone tubing. In certain aspects of the disclosure, the tubing at positions 9 and/or 10 is 14 cm silicone tubing. In some aspects, the vials and/or containers comprise reagents. In some aspects, the vial or container connected to position 9 via tubing contains 2-6 ml of acid (e.g., 4 ml of 1M sulfuric acid). In some aspects, the vial or container connected to position 10 via tubing contains 15-30 ml (e.g., about 20 ml) of phosphate-buffered saline.

In some aspects of the disclosure, position 12 on the cassette contains a vial or container comprising reagents. In some aspects of the disclosure, the vial or container at position 12 comprises a PET tracer precursor dissolved in reagent. In some aspects, the vial or container comprises an FSPG precursor in acetonitrile.

In some aspects of the disclosure, position 14 on the cassette contains a vial or container comprising reagents. In some aspects of the disclosure, the vial or container at position 14 comprises a reagent selected from the group consisting of sulfuric acid, hydrochloric acid, and trifluoroacetic acid. In some aspects of the disclosure, the vial or container at position 14 comprises trifluoroacetic acid. In some aspects of the disclosure, the vial or container at position 14 comprises hydrochloric acid. In some aspects of the disclosure, the vial or container at position 14 comprises sulfuric acid (e.g., 1M sulfuric acid). In some aspects, the vial or container comprises about 1.5 to 2.0 ml of acid (e.g., 1M sulfuric acid). In some aspects, the vial or container comprises 1.7 ml of 1M sulfuric acid. In some aspects, the vial is a 13 mm vial comprising 1.7 ml of 1M sulfuric acid.

In some aspects of the disclosure, position 15 on the cassette contains a means for connecting the cassette to a reagent and/or eluent container. In some aspects, the means for connecting the cassette to a container is a spike. In some aspects, the container is a bag that contains sterile water for injection. In some aspects, the water bag is connected at the spike at position 15.

In some aspects of the disclosure, position 16 on the cassette contains a vial or container comprising reagents. In some aspects of the disclosure, the vial or container at position 16 comprises potassium hydroxide. In some aspects of the disclosure, the vial or container at position 16 comprises sodium hydroxide (e.g., 4 N sodium hydroxide). In some aspects, the vial or container comprises 1.5-2.0 ml (e.g., about 1.7 ml) of 4 N sodium hydroxide. In some aspects, the vial is a 13 mm vial comprising 1.7 ml of 4 N sodium hydroxide.

In some aspects of the disclosure, positions 18 and 20 on the cassette contains a SPE cartridge. In some aspects, the SPE cartridge is a reverse-phase SPE cartridge. Reverse-phase SPE makes use of a nonpolar modified solid phase and a polar mobile phase. For reverse-phase SPE, compounds are retained by hydrophobic interactions and eluted using a non-polar elution solvent to disrupt the forces that bind the compound to the solid phase. Non-limiting examples of reverse-phase SPE cartridges include C18, tC18, CS, CN, Dial, HLB, Porapak, RDX, and NH₂ SPE cartridges. In some aspects, the SPE cartridge has a mixed-mode cation-exchange and reversed-phase sorbent. In some aspects, the reverse-phase/cation exchange SPE cartridge is an Oasis® MCX cartridge. In some aspects of the present disclosure, the reverse-phase SPE cartridge is a tC18 or a HLB SPE cartridge. In some aspects, the reverse-phase SPE cartridge is a HLB SPE cartridge. In another aspect of the present invention the reverse-phase SPE cartridge is a tC18 cartridge. In some aspects of the present invention the tC18 cartridge is an environmental tC18 cartridge, sometimes referred to as a long tC18 cartridge or a tC18 plus cartridge. In some aspects of the disclosure, the reverse-phase SPE cartridge is pre-conditioned with eluent. In some aspects, the reverse-phase or reverse-phase/cation exchange SPE cartridge is pre-conditioned with phosphate-buffered saline or sodium bicarbonate solution. In some aspects, the reverse-phase/cation exchange SPE cartridge is an Oasis® MCX SPE cartridge that has been pre-conditioned with sodium bicarbonate solution.

In some aspects, the SPE cartridge at position 18 is connected to tubing at position 17 of the cassette. In certain aspects of the disclosure, the tubing at position 17 is silicone tubing. In certain aspects of the disclosure, the tubing at position 17 is 12-16 cm (e.g., about 14 cm) silicone tubing.

In some aspects, the SPE cartridge at position 20 is connected to tubing at position 19 of the cassette. In certain aspects of the disclosure, the tubing at position 19 is silicone tubing. In certain aspects of the disclosure, the tubing at position 19 is 12-16 cm (e.g., about 14 cm) silicone tubing.

In some aspects of the disclosure, position 22 on the cassette contains a SPE cartridge. In some aspects, the SPE cartridge is a normal-phase SPE cartridge. Normal-phase SPE makes use of a polar modified solid phase and a non-polar mobile phase. For normal-phase SPE, compounds are retained by hydrophilic interactions and eluted using an eluent that is more polar than the original mobile phase to disrupt the binding mechanism. Non-limiting examples of normal-phase SPE cartridges include alumina, diol, and silica SPE cartridges. In some aspects of the present disclosure, the normal-phase SPE cartridge is an alumina SPE cartridge. In some aspects of the present disclosure, the normal-phase SPE cartridge is a Sep-Pak® Alumina N Plus Long Cartridge. In some aspects, the normal-phase SPE cartridge is pre-conditioned with eluent. In some aspects, the normal-phase SPE cartridge is pre-conditioned with phosphate-buffered saline. In some aspects, the normal-phase SPE cartridge is an alumina SPE cartridge that has been pre-conditioned with phosphate-buffered saline.

In some aspects of the disclosure, position 23 on the cassette contains tubing connected to a SPE cartridge. In certain aspects of the disclosure, the tubing at position 23 is silicone tubing. In certain aspects of the disclosure, the tubing at position 23 is 12-16 cm (e.g., about 14 cm) silicone tubing. In some aspects, the SPE cartridge is a reverse-phase SPE cartridge. In some aspects, the SPE cartridge contains graphitized non-porous carbon. In some aspects the cartridge is an ENVI-carb or Hypercarb cartridge. In some aspects, the SPE cartridge is a Supelco® Superclean™ ENVI-Carb™ or Thermo Fisher Scientific HyperSep™ Hypercarb™ cartridge.

Methods for sterilizing compositions meant for parenteral administration are well known to those skilled in the art. In some aspects, the final [¹⁸F]FSPG solution may be filtered through sterile syringe filter (e.g., with a 0.22 μm pore size) and a mixed cellulose esters membrane before being collected in the final product vial. In some aspects, the final product vial may be subjected to high heat, such as in an autoclave, for example.

### IV. Automated Synthesis Apparatus

Certain aspects of the disclosure are directed to a cassette for use in an automated synthesis apparatus for [¹⁸F]FSPG production. In some aspects, the automated synthesis apparatus a reliable and cGMP-compliant apparatus.

Non-limiting examples of commercially available automated synthesis apparatuses include: GE Healthcare; CTI Inc.; Ion Beam Applications S.A. (Chemin du Cyclotron 3, B-1348 Louvain-La-Neuve, Belgium); Raytest (Germany) and Bioscan (USA).

In some aspects, automated synthesis apparatus comprises a cassette disclose herein. In some aspects, the radiochemistry for [¹⁸F]FSPG production takes place in a cassette or cartridge, which is designed to be removable and fit onto the apparatus. In some aspects, the cassette fits into the apparatus in such a way that mechanical movement of moving parts of the apparatus control the operation of the cassette. In some aspects, the automated synthesis apparatus has a means for connecting to a cyclotron to receive the desired isotope. In some aspects, the automated synthesis apparatus has an external arm, which is capable of rotating, thus controlling the opening or closing of the valve when a suitable cassette is attached to the automated synthesis apparatus. Additional moving parts of the automated synthesis apparatus can be designed to clip onto syringe plunger tips, and thus raise or depress syringe barrels.

### V. Methods for Synthesizing

Certain aspects of the disclosure are directed to a cGMP-compliant process to enable the large-scale production for clinical and commercial use of a [¹⁸F]FSPG radiotracer. Synthesis of [¹⁸F]FSPG on a cassette disclosed herein (e.g., as illustrated by the cassette of FIG. 1) can be carried out by nucleophilic fluorination with [¹⁸F]fluoride produced by a ¹⁸O(p,n)¹⁸F reaction.

In some aspects, [¹⁸F]fluoride suitable for use in the synthesis of the PET tracer can be obtained as an aqueous solution from the nuclear reaction ¹⁸O(p,n)¹⁸F (see, e.g., Hess, E et al 2001, Radiochim. Acta 89, 357-362). In some aspects, a cyclotron is used to produce the [¹⁸F]fluoride. In some aspects, to increase the reactivity of [¹⁸F]fluoride and/or reduce or minimize hydroxylated by-products resulting from the presence of water, water can be removed from [¹⁸F]fluoride prior to the reaction. In some aspects, the fluorination reactions are carried out using anhydrous reaction solvents (see, Aigbirhio et al 1995 J Fluor Chem; 70:279-87). In some aspects, a further step can be used to improve the reactivity of [¹⁸F]fluoride for radiofluorination reactions, comprising adding a cationic counterion prior to the removal of water. This cationic counterion can be dissolved in an organic-aqueous solution and this solution can be used as an eluent for eluting [¹⁸F]fluoride from an anion exchange column on which the [¹⁸F]fluoride has been bound or trapped. In some aspects, the organic-aqueous solution is an aqueous solution of acetonitrile, methanol, and/or water. In some aspects, the organic-aqueous solution is an aqueous solution of acetonitrile. In some aspects, the organic-aqueous solution is an aqueous solution of acetonitrile and water. In some aspects, the counterion can possess sufficient solubility within the anhydrous reaction solvent to maintain the solubility of the [¹⁸F]fluoride. Therefore, counterions that are typically used include large but soft metal ions such as rubidium or cesium, potassium complexed with a cryptand such as Kryptofix™ 222, or tetraalkylammonium salts, wherein potassium complexed with a cryptand such as Kryptofix™ 222, or tetraalkylammonium salts are preferred. The term Kryptofix™ 222 (or K222) refers herein to a commercially-available preparation of the compound 4,7,13,16,21,24-hexaoxa-1,10-diazabicyclo[8.8.8]hexacosane. In some aspects, the potassium is in the form of potassium carbonate. In some aspects, the eluent used for eluting [¹⁸F]fluoride from an anion exchange SPE cartridge on which the [¹⁸F]fluoride has been trapped is a solution of K₂CO₃/Kryptofix™ 222 in 50% water/acetonitrile.

In some aspects, the [¹⁸F]fluoride so-produced enters the cassette at position 6 and is loaded onto a SPE cartridge. In some aspects, the SPE cartridge is a quaternary methyl ammonium anion exchange (QMA) solid phase extraction (SPE) cartridge. In some aspects, the [¹⁸F]fluoride is retained by an ion-exchange reaction in the QMA cartridge and the ¹⁸O-water is allowed to flow through the common pathway of the cassette to be recovered at position 1. In some aspects, [¹⁸F]fluoride retained on the QMA cartridge is then eluted with an eluent solution (e.g., Kryptofix™ 222 and potassium carbonate in acetonitrile at position 2) into the reaction vessel.

In some aspects, the reaction vessel is heated to evaporate the water. In some aspects, the reaction vessel is heated to about 120° C. for 5 to 10 minutes. In some aspects, additional acetonitrile is added and the reaction vessel is heated again to complete the removal of water. In some aspects, the starting activity of the [¹⁸F]fluoride is 0.25-3.5 Ci (10 to 130 GBq). In some aspects, the starting activity of the [¹⁸F]fluoride is 1.0-3.3 Ci (37 to 122 GBq).

In some aspects, the FSPG precursor, dissolved in anhydrous acetonitrile (from position 12), is added to the reaction vessel containing the [¹⁸F]fluoride. In some aspects, 6 mg to 12 mg of FSPG precursor is dissolved in acetonitrile before being adding to the reaction vessel. In some aspects, 6 to 12 mg of FSPG precursor is dissolved in 1.3 ml of acetonitrile. In some aspects, 6 mg of FSPG precursor is dissolved in 1.3 ml anhydrous acetonitrile. In some aspects, the FSPG precursor is di-tert-butyl(2S,4S)-2-(3-((naphthalen-2-ylsulfonyl)oxy) propyl)-4-(tritylamino) pentanedioate or di-tert-butyl (2S,4S)-2-(3-((naphthalen-2-ylsulfonyl)oxy) propyl)-4-(boc-amino) pentanedioate. In some aspects, the FSPG precursor has one of the following chemical structure(s):

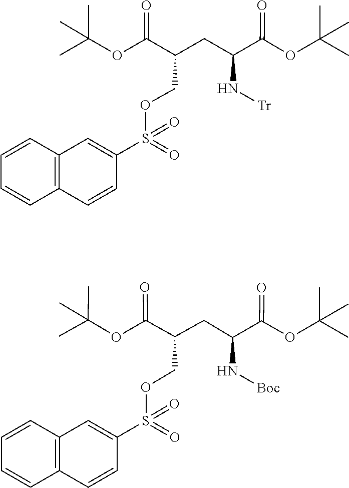

The process for preparing the FSPG precursor is known in the art (see, e.g., Shih et al, PLOS One. 2020; 15 (12): e0243831, which is incorporated herein by reference).

In some aspects, the FSPG precursor has a chemical purity of at least 90%. In some aspects, the FSPG precursor has a chemical purity of at least 95%. In some aspects, the FSPG precursor has a chemical purity of at least 97%. In some aspects, the FSPG precursor has a chemical purity of at least 98%. In some aspects, the FSPG precursor has a chemical purity of at least 99%. In some aspects, 6 mg of di-tert-butyl(2S,4S)-2-(3-((naphthalen-2-ylsulfonyl)oxy) propyl)-4-(tritylamino) pentanedioate or di-tert-butyl (2S,4S)-2-(3-((naphthalen-2-ylsulfonyl)oxy) propyl)-4-(boc-amino) pentanedioate is dissolved in 1.0-1.5 ml (e.g., about 1.3 ml) anhydrous acetonitrile before being added to the reaction vessel.

In some aspects, the reaction vessel is heated at temperature ranging from 100° C. to 120° C. for 5 to 15 minutes to produce the [¹⁸F]-labeled FSPG precursor. In some aspects, the reaction vessel is heated at a temperature of 105° C. to 115° C. for 5 to 15 minutes. In some aspects, the reaction vessel is heated at a temperature of 105° C. to 110° C. for 5 to 15 minutes. In some aspects, the reaction vessel is heated at temperature ranging from 100 to 120° C. for 5 to 10 minutes. In some aspects, the reaction vessel is heated at temperature ranging from 100 to 120° C. for about 5 minutes. In some aspects, the reaction vessel is heated at a temperature of 105° C. for about 5 minutes.

In some aspects, an acid is then added (e.g., from position 14) to the reaction vessel and the mixture is again heated to 105° C. to 115° C. for 2 to 10 minutes. In some aspects, the acid is selected from the group consisting of sulfuric acid, hydrochloric acid, and trifluoroacetic acid. In some aspects, the acid is sulfuric acid. In some aspects, the acid is hydrochloric acid. In some aspects, the acid is trifluoroacetic acid. In some aspects, the reaction vessel is heated at a temperature of 105° C. to 110° C. for 3 to 7 minutes. In some aspects, the reaction vessel is heated at temperature ranging from 100 to 120° C. for 3 to 10 minutes. In some aspects, the reaction vessel is heated at temperature ranging from 100 to 120° C. for 4 minutes. In some aspects, the reaction vessel is heated at a temperature of 105° C. for about 4 minutes.

In some aspects, a base (e.g., from position 16) is then added to the reaction vessel and heated to 60° C. to 80° C. for 5 to 15 minutes to produce the [¹⁸F]-labeled FSPG precursor. In some aspects, the base is sodium hydroxide. In some aspects, the base is potassium hydroxide. In some aspects, the reaction vessel is heated at a temperature of 65° C. to 75° C. for 5 to 15 minutes. In some aspects, the reaction vessel is heated at temperature ranging from 60° C. to 80° C. for 5 to 10 minutes. In some aspects, the reaction vessel is heated at temperature ranging from 65° C. to 75° C. for 5 to 10 minutes. In some aspects, the reaction vessel is heated at a temperature of 70° C. for about 5 minutes.

In some aspects, the pH of the reaction mixture is then increased, i.e., acidified. In some aspects, the pH of the reaction mixture is increased with sulfuric acid, hydrochloric acid, or trifluoroacetic acid. In some aspects, the reaction mixture is acidified with sulfuric acid (e.g., 1 M sulfuric acid). In some aspects, the reaction mixture is acidified with hydrochloric acid. In some aspects, the reaction mixture is acidified with trifluoroacetic acid.

In some aspects, the crude [¹⁸F]FSPG is trapped and separated from the side products on two sequential anion exchange SPE cartridges. In some aspects, the crude [¹⁸F]FSPG is loaded onto the anion exchange SPE cartridges at a flow rate of 8 ml/min to 20 ml/min. In some aspects, the loading flow rate is 10 ml/min to 18 ml/min. In some aspects, the loading flow rate is 12 ml/min to 16 ml/min. In some aspects, the loading flow rate is about 14 ml/min. In some aspects, the cartridges are first washed with water, e.g., to remove side products. In some aspects, the [¹⁸F]FSPG is eluted off the cartridges with a phosphate-buffered saline eluent (e.g., from position 10). In some aspects, the [¹⁸F]FSPG is eluted off the cartridges with a flow rate of 4 ml/min to 16 ml/min. In some aspects, the eluting flow rate is 6 ml/min to 14 ml/min. In some aspects, the eluting flow rate is 8 ml/min to 12 ml/min. In some aspects, the eluting flow rate is about 10 ml/min.

Potential residual [¹⁸F]fluoride removal can take place on an alumina SPE column (e.g., at position 22 via tubing at position 21). The product can be further purified on a normal-phase SPE cartridge (e.g., via tubing at position 23). In some aspects, the product can be sterilized, optionally through a sterile syringe filter (e.g., with a 0.22 μm pore size). In some aspects, the final purified, sterilized product is collected in a receptacle (e.g., a vial). In some aspects, a position (e.g., position 13) of the cassette can be empty. In some aspects, a cap is placed on the valve at the empty position.

In some aspects, the automated process of [¹⁸F]FSPG production disclosed herein takes 10 to 90 minutes. In some aspects, the automated process of [¹⁸F]FSPG production disclosed herein takes 20 to 90 minutes. In some aspects, the automated process of [¹⁸F]FSPG production disclosed herein takes 30 to 90 minutes. In some aspects, the automated process of [¹⁸F]FSPG production disclosed herein takes 10 to 80 minutes. In some aspects, the automated process of [¹⁸F]FSPG production disclosed herein takes 20 to 80 minutes. In some aspects, the automated process of [¹⁸F]FSPG production disclosed herein takes 30 to 80 minutes. In some aspects, the automated process of [¹⁸F]FSPG production disclosed herein takes 10 to 70 minutes. In some aspects, the automated process of [¹⁸F]FSPG production disclosed herein takes 20 to 70 minutes. In some aspects, the automated process of [¹⁸F]FSPG production disclosed herein takes 30 to 70 minutes. In some aspects, the automated process of [¹⁸F]FSPG production disclosed herein takes 10 to 60 minutes. In some aspects, the automated process of [¹⁸F]FSPG production disclosed herein takes 20 to 60 minutes. In some aspects, the automated process of [¹⁸F]FSPG production disclosed herein takes 30 to 60 minutes. In some aspects, the automated process of [¹⁸F]FSPG production disclosed herein takes 10 to 50 minutes. In some aspects, the automated process of [¹⁸F]FSPG production disclosed herein takes 20 to 50 minutes. In some aspects, the automated process of [¹⁸F]FSPG production disclosed herein takes 30 to 50 minutes.

In some aspects, the automated process of [¹⁸F]FSPG production disclosed herein takes less than 90 minutes. In some aspects, the automated process of [¹⁸F]FSPG production disclosed herein takes less than 80 minutes. In some aspects, the automated process of [¹⁸F]FSPG production disclosed herein takes less than 70 minutes. In some aspects, the automated process of [¹⁸F]FSPG production disclosed herein takes less than 60 minutes. In some aspects, the automated process of [¹⁸F]FSPG production disclosed herein takes less than 50 minutes. In some aspects, the automated process of [¹⁸F]FSPG production disclosed herein takes less than 40 minutes.

### EXAMPLES

### Example 1. Automated Process for Radiolabeling FSPG

The table below details the cassette positions for the automated radiosynthesis of ¹⁸F-FSPG. The cassette positions correspond to the labeled positions of the cassette shown in FIG. 1.

| Cassette Position | Component |
|---|---|
| 1 | 14 cm silicone tubing connected to ¹⁸O- |
| | water collection vial |
| 2 | Potassium carbonate/Kryptofix elution |
| | solution for QMA cartridge(11 mm vial, |
| | 0.8 mL) |
| 3 | Syringe 1 |
| 4 | Pre-conditioned QMA cartridge |
| 5 | 14 cm silicone tubing to the QMA |
| | cartridge at position 4 |
| 6 | ¹⁸F receiving inlet |
| 7 | 14 cm silicone tubing connected to the |
| | left side of reactor |
| 8 | 14 cm silicone tubing connected to the |
| | center of reactor |
| 9 | 42 cm silicone tubing connected to vial of |
| | 1M sulfuric acid |
| 10 | 42 cm silicone tubing connected to vial of |
| | PBS solution |
| 11 | Syringe 2 |
| 12 | Precursor in acetonitrile |
| 13 | Empty |
| 14 | 1M Sulfuric acid (13 mm vial, 2.2 mL) |
| 15 | Water bag spike |
| 16 | 4N Sodium hydroxide (13 mm vial, 1.7 |
| | mL) |
| 17 | 14 cm silicone tubing to the MCX |
| | cartridge at position 18 |
| 18 | MCX cartridge (pre-conditioned with 10 |
| | mL PBS) |
| 19 | 14 cm silicone tubing to the MCX |
| | cartridge at position 20 |
| 20 | MCX cartridge (pre-conditioned with 10 |
| | mL PBS) |
| 21 | 14 cm silicone tubing to the Sep-Pak |
| | Alumina N Plus Long cartridge at |
| | position 22 |
| 22 | Sep-Pak Alumina N Plus Long cartridge |
| | (pre-conditioned with 10 mL PBS) |
| 23 | 42 cm silicone tubing connected to an |
| | ENVI-Carb cartridge |
| 24 | Syringe 3 |
| 25 | 42 cm silicone tubing to the right side of |
| | reactor |

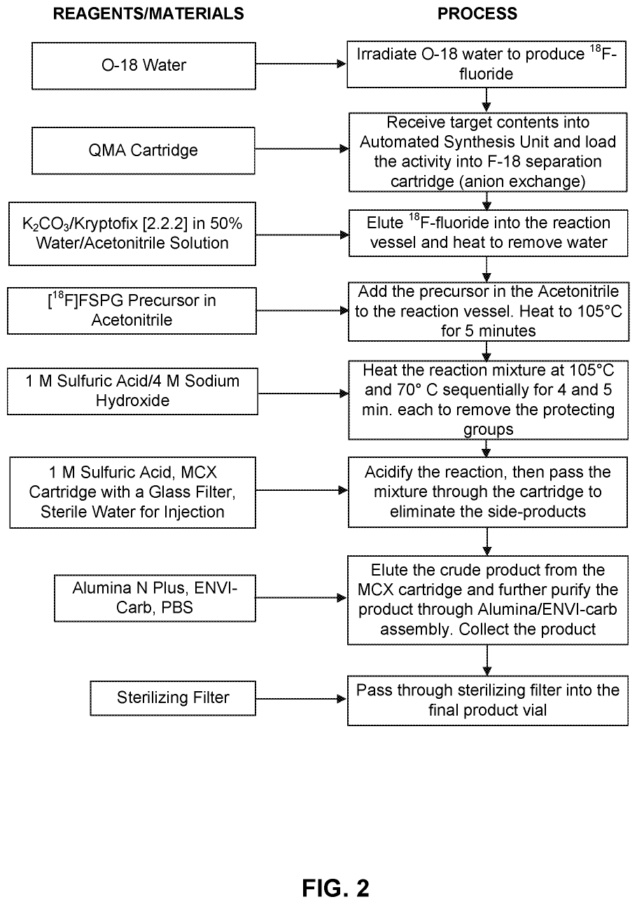

FIG. 2 illustrates the flow diagram of the automated manufacturing process for radiolabeling FSPG. The cassette configuration as illustrated in FIG. 1 was used to produce [¹⁸F]FSPG using the following method (numbers in this method are reference numbers in FIG. 1 unless stated as a “position”, which is one of positions 1-25 going from left to right on the cassette of FIG. 1):
(i) [¹⁸F] Fluoride was obtained from the bombardment of [¹⁸O]—H₂O with a high-energy proton beam extracted from a PET Trace (GE Healthcare) and transferred to the cassette via the conical reservoir at position 6.
(ii) [¹⁸F] Fluoride was trapped on the QMA column at position and separated from the enriched water which was collected in an external vial via a pathway through positions 5-4-1.
(iv) Eluent containing potassium carbonate and Kryptofix™ 222 in 50% water/acetonitrile (from the vial at position 2) was withdrawn in the syringe at position 3 and passed through the QMA cartridge to release [¹⁸F]fluoride and send to the reaction vessel.
(v) Evaporation of the water in the reaction vessel was catalyzed by heating the reaction vessel at 120° C. for 5 minutes to complete the removal of water.
(vi) 6 to 12 mg of the FSPG precursor in 1.3 ml of anhydrous acetonitrile is added to the reaction vessel from the vial at position 12. The labelling reaction was carried out by heating the reaction vessel at 105° C. for 5 minutes.
(vii) A 2.2 ml first aliquot of 1M sulfuric acid from the vial at position 14 is added to the reaction vessel and the reaction vessel is heated to 105° C. for 4 minutes to remove the protecting groups from the radiolabeled precursor.
(viii) A 1.7 ml aliquot of 4M sodium hydroxide solution from the vial at position 16 is added to the reaction vessel and the reaction vessel is heated to 70° C. for 5 minutes.
(ix) The reaction mixture is added to the 10 mL vial at position 9 containing 4 mL of 1M sulfuric acid to acidify the solution.
(x) The resulting crude [¹⁸F]FSPG was loaded at a rate of 14 ml/min and trapped on two Oasis® MCX cartridges at position 18 via tubing at position 17 and position 20 via tubing at position 19. The cartridges are first washed with water to remove side products. The [¹⁸F]FSPG is eluted off the cartridges with PBS eluent (from position 10) at a rate of 10 ml/min, which is passed in the same direction as the crude [¹⁸F]FSPG was loaded onto the cartridges.
(xi) The [¹⁸F]FSPG solution was then passed through an alumina SPE column at position 22 via tubing at position 21 to remove residual [¹⁸F]fluoride.
(x) The final product was passed through an external ENVI-carb cartridge connected to the cassette by tubing at position 23. The final product was then passed through a sterile syringe filter with a 0.22 μm pore size before being collected in a sterile final product vial.

This cassette configuration has an enriched water recycling pathway on the cassette (contamination of the manifold with enriched water possible) with four positions on the cassette engaged, i.e. position 6 for the activity inlet, position 1 with the connection of enriched water vial, position 4 for the QMA cartridge and position 5 for tubing of QMA cartridge.

Starting activity, final activity and residual activities were measured by a calibrated ionization chamber CAPINTEC (CRC25-PET). To determine yield, the following yield Calculations were made:
delta Tf=elapsed time after time at starting of the synthesis in min
Af=final activity in mCi
cAf=corrected final activity in mCi with respect to the start of the synthesis in min=Af*Exp(ln(2)*(delta Tf/110)) where 110=half-life of [¹⁸F]fluorine in minutes cAi=corrected starting activity in mCi regarding to starting of the synthesis in mCi delta Ts=duration of the synthesis

$$Corrected yield ( C Y ) = ( cAf / cAi ) * 100$$

$$Uncorrected yield ( NCY ) = C Y * Exp ( ln ( 2 ) * ( - delta Ts / 110 ) )$$

A [¹⁸F]FSPG composition was prepared according to the disclosed method. The [¹⁸F]FSPG composition prepared according to the disclosed method had the following characteristics:

$$Starting Activity = 1. - 3.3 Ci$$

$$Final Concentration = 7. - 52. mCi / ml$$

$$pH = 6. to 8.$$

$$Radiochemical Yield = 18 \% - 32 \% , non - decay corrected$$

The radiochemical purity of the [¹⁸F]FSPG composition was tested at the end of synthesis and up to 5 hours after being stored at room temperature. Analytical reverse phase-HPLC was performed on an Agilent 1260 HPLC system equipped with a 1200 Series Diode Array Detector and a Raytest GABI Star NaI (TL) scintillation detector. The following process parameters were used:
Column: Phenomenex Luna C-18 (2) 250 mm×4.6 mm (5 μm)
Mobile Phase A: 40 μM aqueous disodium phosphate
Mobile Phase B: Acetonitrile/Methanol/Water (45:45:10) (% w/v)
Flow rate: 1.5 ml/min
Gradient:
   Initial: 100% Mobile Phase A, 0% Mobile Phase B
   2 min: 96% Mobile Phase A, 4% Mobile Phase B
   5 min: 88% Mobile Phase A, 12% Mobile Phase B
   15 min: 88% Mobile Phase A, 12% Mobile Phase B
   20 min: 40% Mobile Phase A, 60% Mobile Phase B
   22 min: 0% Mobile Phase A, 100% Mobile Phase B
   24 min: 0% Mobile Phase A, 100% Mobile Phase B
   25 min: 100% Mobile Phase A, 0% Mobile Phase B
   30 min: 100% Mobile Phase A, 0% Mobile Phase B
Total Run Time: 35 minutes
UV detector: 340 nm
[¹⁸F]FSPG solution (80 μl) was mixed with o-phthaldialdehyde reagent solution (20 μl). The mixture sat for 1 min prior to the HPLC analysis.

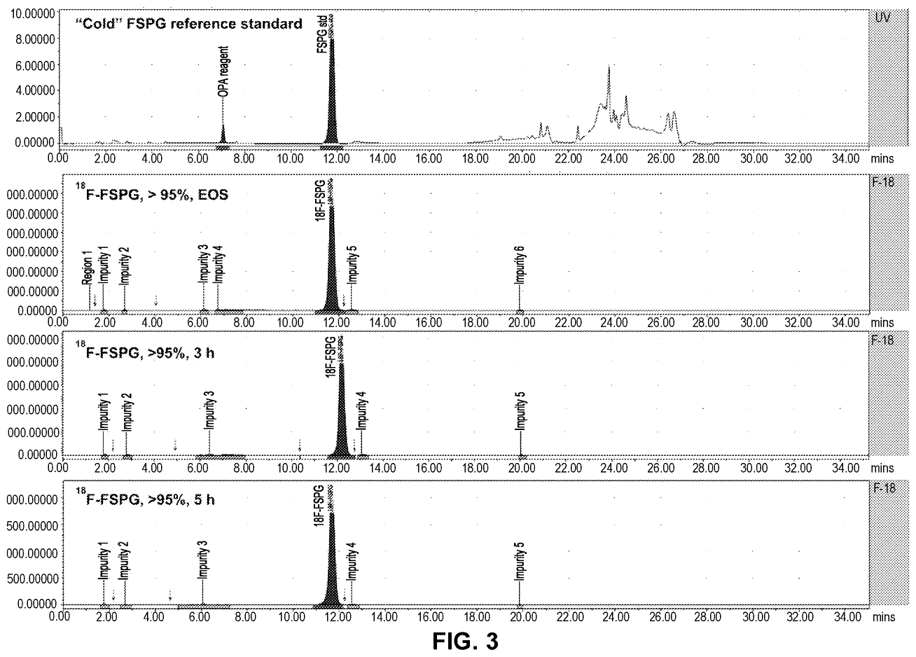

At the end of synthesis, the radiochemical purity of the [¹⁸F]FSPG composition was >95%. When stored at room temperature, the [¹⁸F]FSPG composition maintained a radiochemical purity of >95% for 5 hours (FIG. 3).

What is claimed is:

1. An automated process for the synthesis of (S)-4-(3-¹⁸F-fluoropropyl)-L-glutamic acid ([¹⁸F]FSPG) in a cassette, the method comprising:
a. Dissolving a (S)-4-(3-¹⁸F-fluoropropyl)-L-glutamic acid precursor in anhydrous acetonitrile and adding the resulting solution to a reaction vessel comprising radiolabeled fluoride (¹⁸F);
b. Heating the reaction vessel at 100° C. to 110° C. for 5 minutes to 15 minutes to provide a crude intermediate product;
c. Adding a first aliquot of an acid to the reaction vessel and heating at 100° C. to 110° C. for 2 to 10 minutes followed by adding an aliquot of a base to the reaction vessel and heating at 60° C. to 80° C. for 5 to 15 minutes to remove protecting groups to provide a crude [¹⁸F]FSPG;
d. Acidifying the crude [¹⁸F]FSPG;
e. Loading the acidified crude ¹⁸F-FSPG onto one or more reverse-phase/cation exchange solid phase extraction cartridges;
f. Eluting the [¹⁸F]FSPG off the one or more reverse-phase solid phase extraction cartridges;
g. Purifying the [¹⁸F]FSPG through one or more carbon-phase/reverse-phase solid phase extraction cartridges; and
wherein each of (a)-(g) are within the cassette.

2. The process according to claim 1, where the [¹⁸F]FSPG is eluted off the one or more reverse-phase solid phase extraction cartridges with a buffer.

3. The process according to claim 2, wherein the buffer is phosphate-buffered saline.

4. The process according to any preceding claims, wherein the first aliquot of acid in step (c) is selected from the group consisting of sulfuric acid, hydrochloric acid, and trifluoroacetic acid.

5. The process according to any preceding claims, wherein the first aliquot of acid in step (c) is sulfuric acid.

6. The process according to any preceding claims, wherein the first aliquot of acid in step (c) is hydrochloric acid.

7. The process according to claim 1 or 2, wherein the first aliquot of acid in step (c) is trifluoroacetic acid.

8. The process according to any preceding claims, wherein the base in step (c) is sodium hydroxide.

9. The process according to any preceding claims, wherein the base in step (c) is potassium hydroxide.

10. The process according to any preceding claim, wherein the one or more reverse-phase solid phase extraction cartridges are washed with water to remove side products before step (f).

11. The process according to any preceding claim, wherein the crude [¹⁸F]FSPG is acidified with a second aliquot of sulfuric acid in step (d).

12. The process according to any preceding claim, further comprising purifying the [¹⁸F]FSPG through one or more normal-phase solid phase extraction cartridges outside of the cassette.

13. The process according to any preceding claim, further comprising sterilizing the [¹⁸F]FSPG product through a sterilizing filter to produce a final [¹⁸F]FSPG composition.

14. The process according to any preceding claim, wherein the final purified [¹⁸F]FSPG has a greater than 90% radiochemical purity.

15. The process according to any preceding claim, wherein the final purified [¹⁸F]FSPG has a greater than 95% radiochemical purity.

16. The process according to any preceding claim, wherein the final purified [¹⁸F]FSPG has a greater than 96% radiochemical purity.

17. The process according to any preceding claim, wherein the final purified [¹⁸F]FSPG has a greater than 97% radiochemical purity.

18. The process according to any preceding claim, wherein the final purified [¹⁸F]FSPG has a greater than 98% radiochemical purity.

19. The process according to any preceding claim, wherein the process produces [¹⁸F]FSPG with a non-decay corrected radiochemical yield of at least 10%.

20. The process according to claim 19, wherein the process produces [¹⁸F]FSPG with a non-decay corrected radiochemical yield of 13% to 35%.

21. The process according to claim 19, wherein the process produces [¹⁸F]FSPG with a non-decay corrected radiochemical yield 18% to 32%.

22. The process according to any preceding claim, wherein the reaction product is loaded onto the reverse-phase solid phase extraction with a flow rate of 8 ml/min to 20 ml/min and eluted off the reverse-phase solid phase extraction cartridge with a flow rate of 4 ml/min to 14 ml/min.

23. The process according to claim 22, wherein the reaction product is loaded onto the reverse-phase solid phase extraction with a flow rate of 14 ml/min and eluted off the reverse-phase solid phase extraction cartridge with a flow rate of 10 ml/min.

24. The process according to any preceding claim, wherein the one or more solid phase extraction cartridges in step (e) contain reverse-phase/cation exchange sorbents.

25. The process according to any preceding claim, wherein the [¹⁸F]FSPG is eluted off the reverse-phase solid phase extraction cartridge in the same direction as the crude [¹⁸F]FSPG was loaded.

26. The process according to any preceding claim, wherein the one or more reverse-phase solid extraction cartridges in step (f) comprise aluminum oxide.

27. The process according to any preceding claim, wherein the one or more carbon-phase/reverse-phase solid extraction cartridges in step (g) comprise a carbon sorbent.

28. The process according to any preceding claim, wherein the FSPG precursor has one of the following structure(s):

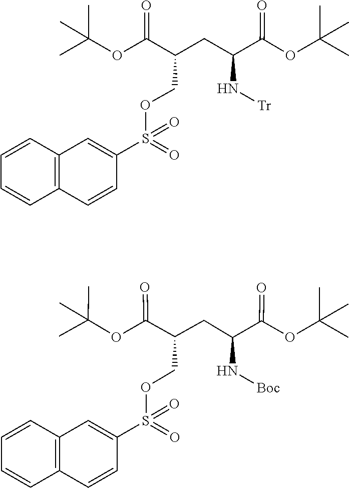

29. The process according to any preceding claim, wherein the [¹⁸F]FSPG has a starting activity of about 10 to 125 Gbq.

30. The process according to any preceding claim, wherein the final [¹⁸F]FSPG composition has a concentration of about 185 to 1850 MBq/ml.

31. The process according to any preceding claim, wherein the final [¹⁸F]FSPG composition is produced in less than 40 minutes from the start of synthesis.

32. The process according to any preceding claim, wherein the final [¹⁸F]FSPG composition has a pH of 6.5 to 7.5.

33. The process according to any preceding claim, wherein the radiolabeled fluoride is prepared by a method comprising:
a. Irradiating ¹⁸O water to produce a solution of ¹⁸F in ¹⁸O water;
b. Loading the ¹⁸F solution onto a quaternary ammonium anion exchange cartridge pre-conditioned with potassium bicarbonate;
c. Eluting ¹⁸F from the cartridge into the reaction vessel with an eluent of potassium carbonate/4,7,13,16,21,24-hexaoxa-1,10-diazabicyclo[8.8.8]hexacosane in 50% water/acetonitrile solution; and
d. Heating the reaction vessel to remove water.

34. The process according to claim 33, wherein the reaction vessel is heated to 120° C. for about 5 minutes in step (d).

35. A composition comprising [¹⁸F]FSPG prepared by the process of any of claims 1-34.

36. The composition according to claim 35, in which the composition does not undergo radiolysis for at least 1 hour after synthesis when stored at room temperature.

37. The composition according to claim 35, in which the composition does not undergo radiolysis for at least 2 hours after synthesis when stored at room temperature.

38. The composition according to claim 35, in which the composition does not undergo radiolysis for at least 3 hours after synthesis when stored at room temperature.

39. The composition according to claim 35, in which the composition does not undergo radiolysis for at least 4 hours after synthesis when stored at room temperature.

40. The composition according to claim 35, in which the composition does not undergo radiolysis for at least 5 hours after synthesis when stored at room temperature.

41. The composition according to claim 35, in which the composition does not undergo radiolysis for at least 6 hours, 7 hours, or 8 hours after synthesis when stored at room temperature.

42. The composition according to claim 35, in which the composition maintains a greater than 90% radiochemical purity for at least 5 hours after storage at room temperature.

43. A composition according to claim 35, in which the composition maintains a greater than 95% radiochemical purity for at least 5 hours after storage at room temperature.

44. A composition according to claim 35, in which the composition maintains a greater than 95% radiochemical purity for at least 6 hours, 7 hours, or 8 hours after storage at room temperature.

45. The process according to claim 33, wherein the quaternary ammonium anion exchange cartridge is pre-conditioned with potassium bicarbonate.
